(19) United States
(12) Patent Application Publication
(10) Pub. No.: US 2015/0058182 A1
(43) Pub. Date: Feb. 26, 2015

(54) MANAGEMENT OF PHARMACY ITEMS BASED ON CONTEXTUAL ATTRIBUTES

(71) Applicant: KIT CHECK, INC., Washington, DC (US)

(72) Inventors: Timothy James Leo Kress-Spatz, Washington, DC (US); Kevin William MacDonald, Washington, DC (US); Nicholas Bastien Petersen, Washington, DC (US); Michael David Waud, Washington, DC (US)

(21) Appl. No.: 14/126,419

(22) PCT Filed: Dec. 13, 2013
(86) PCT No.: PCT/US13/74872

§ 371 Date: Dec. 14, 2013

Related U.S. Application Data
(60) Provisional application No. 61/868,395, filed on Aug. 21, 2013.

(51) Int. Cl. G06Q 10/08; G06Q 50/22

(52) U.S. Cl. CPC G06Q 10/087; G06Q 50/22
USPC 705/28

(57) ABSTRACT

A method of operating an electronic system comprises receiving an input identifying one or more pharmacy items, accessing stored information corresponding to the one or more pharmacy items based on the received input, determining a value of at least one contextual attribute of the one or more pharmacy items based on the accessed information, and selectively executing, blocking, or modifying a workflow to control distribution of the one or more pharmacy items according to the value of the at least one contextual attribute.

### CROSS-REFERENCE TO RELATED APPLICATION

This application claims priority under 35 U.S.C. §119 to U.S. Provisional Application No. 61/868,395 filed on Aug. 21, 2013, the subject matter of which is hereby incorporated by reference.

### BACKGROUND

The distribution of pharmacy items is governed, in many contexts, by regulations that require qualified professionals to inspect and verify the items. For example, in hospital pharmacies, state regulations generally require a technician and/or pharmacist to inspect and verify the contents and labeling of pharmacy items for accuracy before they are sent out for use by a physician or patient. These inspection and verification procedures are intended to prevent a pharmacy from dispensing erroneously labeled or otherwise defective items that may pose a danger to end users.

In a conventional inspection and verification procedure, a technician or pharmacist visually inspects a pharmacy item or collection of items and then completes appropriate paperwork and/or data entry to confirm the that inspected items contain the proper contents and are accurately labeled. Once the items are verified, they can be dispatched to their destination, such as a designated location in a hospital.

Among other shortcomings, conventional inspection and verification procedures tend to be relatively slow and error prone. As to their slowness, these procedures generally require a qualified professional to look at each item and read the corresponding label, one by one. This can be time consuming, both because hospital pharmacies tend to dispense numerous items in the ordinary course of business, and because the inspection of each item may require attention to fine labeling details, such as differing expiration dates and product names, among other things. Moreover, these procedures are usually performed by highly trained pharmacy staff, which may be an ineffective use of their time. As to being error prone, conventional procedures suffer from basic human errors, such as misreading a label, failing to account for certain items, failing to appropriately document the inspection, or failing to take appropriate actions based on the inspection. The consequences of such human error can be significant, as the misreading of even one item may result in a patient receiving the wrong medication.

Due to these and other shortcomings, there is a general need for improved techniques and technologies for inspecting and verifying pharmacy items.

### SUMMARY

In one embodiment of the inventive concept, a method of operating an electronic system comprises receiving an input identifying one or more pharmacy items, accessing stored information corresponding to the one or more pharmacy items based on the received input, determining a value of at least one contextual attribute of the one or more pharmacy items based on the accessed information, and selectively executing, blocking, or modifying a workflow to control distribution of the one or more pharmacy items according to the value of the at least one contextual attribute.

In another embodiment of the inventive concept, a system comprises an input capture unit configured to receive an input identifying one or more pharmacy items, a contextual attribute identification unit configured to access stored information corresponding to the one or more pharmacy items based on the received input, and further configured to determine a value of at least one contextual attribute of the one or more pharmacy items based on the accessed information, and a workflow control component configured to selectively execute, block, or modify a workflow to control distribution of the one or more pharmacy items according to the value of the at least one contextual attribute.

### BRIEF DESCRIPTION OF THE DRAWINGS

The drawings illustrate selected embodiments of the inventive concept. In the drawings, like reference numbers indicate like features.

FIG. 1 is a block diagram of a system for managing pharmacy items based on contextual attributes, according to an embodiment of the inventive concept.

FIG. 2 is a flowchart illustrating a method of managing pharmacy items based on contextual attributes, according to an embodiment of the inventive concept.

FIG. 3 is a workflow diagram illustrating a method of blocking the distribution of a pharmacy kit containing unverified items, according to an embodiment of the inventive concept.

FIG. 4 is a workflow diagram illustrating a method of verifying multiple pharmacy items in batch mode, according to an embodiment of the inventive concept.

FIG. 5 shows a display interface confirming verification of a pharmacy item, according to an embodiment of the inventive concept.

FIG. 6 shows a display interface allowing input to verify a pharmacy item, according to an embodiment of the inventive concept.

FIG. 7 shows a display interface allowing input to verify multiple pharmacy items in batch mode, according to an embodiment of the inventive concept.

FIG. 8 shows a display interface with information used to verify a pharmacy item, according to an embodiment of the inventive concept.

FIG. 9 shows a display interface providing a warning that a scanned pharmacy kit contains unverified items, according to an embodiment of the inventive concept.

### DETAILED DESCRIPTION

Embodiments of the inventive concept are described below with reference to the accompanying drawings. These embodiments are presented as teaching examples and should not be construed to limit the scope of the inventive concept.

The described embodiments relate generally to management of pharmacy items based on contextual attributes associated with the items. For example, in certain embodiments, one or more pharmacy items are inspected to identify one or more of their contextual attributes, and then a workflow associated with the items is executed, modified, or blocked based on the contextual attributes. As will be apparent from the following description, pharmacy items, workflows, and workflow execution, modification, or blocking can take many alternative forms.

In certain embodiments, pharmacy items are processed in several different stages before being distributed to locations inside a hospital such as smaller satellite pharmacies, dispensing stations, or pre-prepared groups of medications in kits and trays. A system as described below may halt or alter the process of advancing a kit, tray or bulk group of medications in the event that contextual attributes of the item do not meet the requirements of a rules engine evaluating the item's appropriateness for processing. For example, suppose a box of 24 vials of dopamine had been entered into inventory and tagged with radio frequency identification (RFID) tags. Where a user attempts to include one of those vials into a tray destined for use in an operating room, the system may evaluate every item in that tray and, finding that one of the vials had not been verified, stop the process of completing the tray and require an authorized user to verify the item or remove it from the tray before tray completion would be allowed.

To accomplish this, each pharmacy item may have a series of contextual attributes assigned to it, and those contextual attributes may be evaluated by the rules engine, which can be any functional apparatus capable of evaluating conditions related to the contextual attributes. Where an item is received into inventory, the system assigns it a discrete identifier in a database of all labeled items at that facility. This identifier may be programmed into an RFID tag that can be affixed to the container for the item. A database entry for that identifier may include other distinguishing information about the item, including what pharmaceutical product it represents, what lot or batch of that product it came from, a serial number, when it expires, how long it can stay unrefrigerated, what physical location it is stored in, and whether or not it has been verified by a qualified user.

The process of changing an item's status to indicate that an acceptable verification process had been followed could be accomplished in various alternative ways. For example, a batch processing option in which entire boxes or trays of unverified medications could be received into inventory through the use of a RFID scanning station that reads the discrete identifier off of each tag affixed to a medication in the reader and allows for bulk updates of the status to be made is perhaps the easiest. However, other methods of accomplishing this could include using an optical scanner or typing in the unique item identifier in order to update single items requiring status updates. In some embodiments, a first user performs an initial scan to enter an item into inventory, resulting in a verification attribute of the item being set to a status indicating it has not been verified. Thereafter, a second user who is authorized to perform verification changes the verification attribute on the item to indicate that it has been verified.

Where the system is able to distinguish between received items and verified items, a pharmacy could use computerized reporting tools to investigate the amount of received, verified, deployed and used inventory they have recorded, and manage the process of accepting new items into inventory and deploying them out to satellite pharmacy locations, patient care areas, or medications kits and trays.

As used herein, the term “pharmacy item” (or simply “item”) refers to any type of item that is formally dispensed or distributed by way of a pharmacy. In a typical context, such items may include medications, medical instruments, and other materials used by medical professionals to treat patients. The more specific term “regulated pharmacy item” refers to a pharmacy item whose dispensing or distribution is governed by rules of inspection and verification, with such rules being promulgated by, e.g., a state pharmacy board or the hospital itself. In various alternative embodiments, pharmacy items may be processed individually or in groups, referred to as “pharmacy kits”. For instance, inspection and verification procedures may be applied to a kit as a whole, or to individual items independent of a kit. In this context, the term “pharmacy kit” or “kit” denotes a group of items specified by a template. Examples of pharmacy kits, as well as various properties and management techniques for kits, are disclosed in U.S. patent application Ser. No. 13/554,342 filed Jul. 20, 2012, the subject matter of which is hereby incorporated by reference.

As used herein, the term “contextual attribute” refers to information that has been expressly associated with a pharmacy item or collection of items through a handling process such as labeling, data entry, scanning, tracking, or updating, for example. Contextual attributes may include, for instance, a verification status, current and/or previous item handlers, current and/or previous location(s), associated items, previous workflow steps performed in relation to the item, and so on. The value of a contextual attribute may be determined according to human and/or machine interaction with an item or items. For example, a medical professional may identify and record a contextual attribute by manually entering it into a computer, or the contextual attribute may be identified and recorded by a machine that scans a tag or label on the item. Some ways to associate a contextual attribute with an item include, for instance, linking the attribute and item together in database, or recording the attribute information in a label affixed to the item.

As used herein, the term “workflow” denotes a predetermined sequence of steps that governs the dispensing or distribution of pharmacy items. In general, it may refer to processes performed by humans, electronic systems, and/or various other forms of available equipment. For example, a workflow may comprise a pharmacist scanning a pharmacy item for verification, followed by a computer displaying and/or printing information related to the verification, and subsequent dispatching of the pharmacy item to a destination within a hospital. Typically, the ongoing execution of a workflow is controlled by way of an electronic system, such as a computer network, which may display or record information necessary to perform various processes.

A workflow may be said to be “blocked” where predetermined steps of the workflow are prevented from being performed due to contingencies in the workflow. For instance, where the workflow includes steps for verifying an item and then dispatching the item contingent upon successful verification, the dispatching and any subsequent steps may be prevented or “blocked” upon failed verification. The blocking of a workflow may be accompanied by additional steps, such as displaying a warning screen indicating that a problem has occurred during verification and providing information to fix the problem. Similarly, a workflow may be said to be “modified” where steps are added to or removed from the workflow due to contingencies in the workflow. For instance, where the workflow determines a location of an item, it may add or remove steps depending on the location. A workflow is said to be “executed” where its predetermined or modified steps are performed. In various alternative embodiments, the modification of a workflow may take the form of displaying various screens to a user. For instance, modifying a workflow may involve displaying a first screen to a user if an item is determined to be located in the pharmacy, or displaying a second screen to the user if the item is determined to be located in another location of a hospital.

Certain portions of a workflow, such as verification, may require actions to be performed by an “authorized professional”. Such a professional may include, for instance, a pharmacist, a qualified technician, or some other person indicated by a relevant regulation. The regulation may include, for example, those having the force of law at a federal, state, or local level, or those stipulated by a medical facility's self imposed rules. In general, actions performed by an authorized professional may be accompanied by some form of logging to confirm such a professional's involvement. Such logging may comprise, for instance, signing a printed charge sheet or entering confirmation data for verified items.

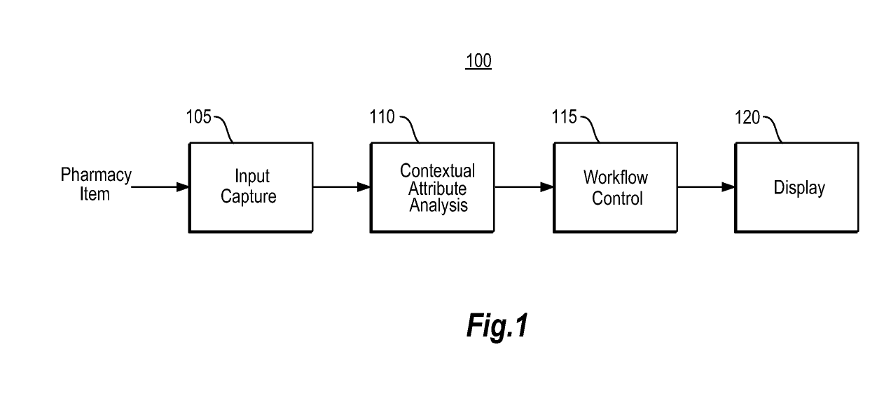

FIG. 1 is a block diagram of a system 100 for managing pharmacy items based on contextual attributes, according to an embodiment of the inventive concept. System 100 is typically implemented by a computer system and associated peripheral devices, but it can take other forms. In a typical hospital setting, multiple instances of system 100, or portions thereof, may be located at different parts of the hospital so that pharmacy items can be managed at different checkpoints.

Referring to FIG. 1, system 100 comprises an input capture component 105, a contextual attribute analysis component 110, a workflow control component 115, and a display component 120. Collectively, these components are designed to capture information for pharmacy items and to manage a workflow of the pharmacy items based on the captured information. Each of these components is typically implemented by a combination of hardware and software.

Input capture component 105 typically comprises an RFID scanner or bar code scanner capable of capturing information from one or more pharmacy items. Such a device may operate automatically in response to the presence of one or more pharmacy items (e.g., by detecting and interrogating RFID tags), or it may operate in response to some form of user initiation, such as the pressing of a button, the handling of a bar code or RFID scanner, or the operation of a computer user interface. Input capture component 105 may operate in batch mode to scan multiple items concurrently or in succession, or it may capture information for one item at a time. In addition, input capture component 105 may comprise more than one device. For instance, it may comprise a collection of different input devices for capturing relevant information used to manage the pharmacy items.

During typical operation, a user presents one or more pharmacy items to input capture component 105. Upon recognizing the presence of the one or more pharmacy items, input capture component 105 receives input data identifying the one or more items and transmits the input data to contextual attribute analysis component 110. Such input data can be, for instance, information encoded in a bar code label or RFID tag affixed to the one or more pharmacy items.

In addition to receiving the input data identifying the one or more pharmacy items, input capture component 105 may also receive information used to update contextual attributes of the pharmacy items. For example, when a user scans an item, the user may also supply information indicating who performed the scanning, whether the item has been verified, and so on.

Contextual attribute analysis component 110 accesses stored information corresponding to the one or more pharmacy items based on the received input data, and it determines a value of at least one contextual attribute of the one or more pharmacy items based on the accessed information. The stored information may be located in a database used by an institution to manage a collection of items. For instance, a hospital may use a central database to track pharmacy items throughout the hospital. Among other things, the contextual attribute may indicate whether labeling and contents of the one or more pharmacy items have been verified by one or more authorized pharmacy professionals, it may indicate a current or prior location of the one or more pharmacy items, or it may indicate a current or prior handler of the one or more pharmacy items. Additionally, where the one or more pharmacy items constitute a pharmacy kit, the at least one contextual attribute may correspond to the pharmacy kit as a whole, e.g., whether the kit has been verified, a location of the kit, etc.

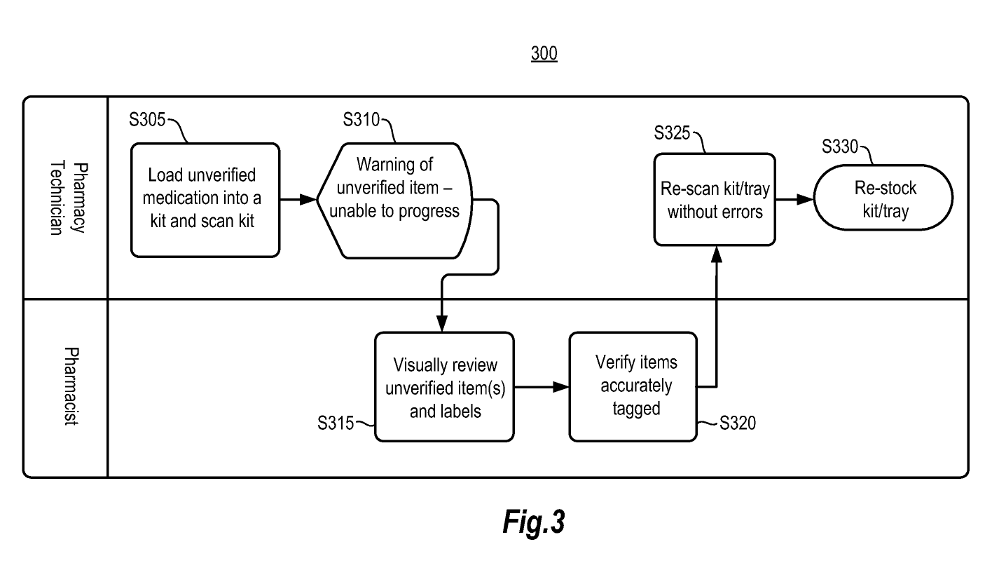

Workflow control component 115 selectively executes, blocks, or modifies a workflow to control distribution of the one or more pharmacy items according to the identified value of the at least one contextual attribute. Various examples of the selective execution, blocking, or modification are described below with reference to FIGS. 3 through 9.

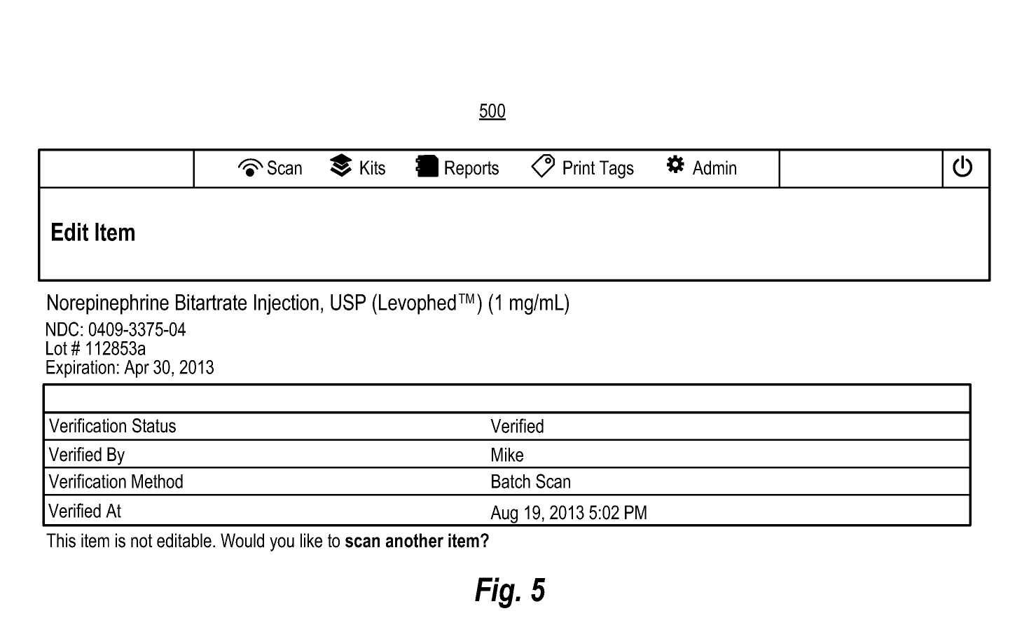

Display component 120 displays information related to the workflow. For example, it may display various screens indicating the values of contextual attributes, screens confirming or prompting inspection and verification of pharmacy items, screens providing warnings of failed verification, and so on. Display component 120 may present screens for entering data related to verification or contextual attributes. Various examples of information to be displayed on display component 120 are illustrated in FIGS. 5 through 9. In addition to display component 120, system 100 may provide other forms of output, such a printed charge sheet produced upon successful validation of one or more pharmacy items, or audio feedback such as alerts in the workflow.

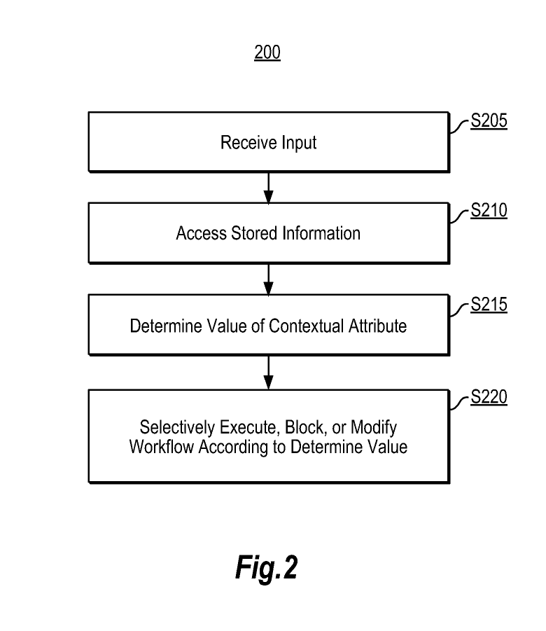

FIG. 2 is a flowchart illustrating a method 200 of managing pharmacy items based on contextual attributes, according to an embodiment of the inventive concept. The method of FIG. 2 could be performed by system 100, for instance, although it is not limited to this or any other particular system implementation.

Referring to FIG. 2, the method comprises receiving an input identifying one or more pharmacy items (S205). As indicated above, such an input may be received through an automated or quasi-automated process such as bar code scanning or RFID scanning. It can also be received through manual user input.

The method further comprises accessing stored information corresponding to the one or more pharmacy items based on the received input (S210), and determining a value of at least one contextual attribute of the one or more pharmacy items based on the accessed information (S215). These steps may be performed, for instance, by accessing a database containing values of contextual attributes for a large collection of items that have been registered with a system or institution. It may also involve analysis of information encoded in a bar code or RFID tag.

Finally, the method comprises selectively executing, blocking, or modifying a workflow to control distribution of the one or more pharmacy items according to the value of the at least one contextual attribute (S220). As one example of this step, if the at least one contextual attribute indicates that a drug is expired or recalled, the method may block a user from performing a subsequent step of a workflow and/or display a warning. As another example, if the at least one contextual attribute indicates that a drug has already been used on a patient, the method may display information indicating the presence of a new patient and it may therefore prevent a user administering the drug to the new patient. As yet another example, if the at least one contextual attribute indicates that a drug is considered to be a substitute for another drug or has “abnormal strength”, the method may allow a user to continue a workflow but may also display a warning indicating this is a change from normal procedure.

As still another example, if the at least one contextual attribute indicates that an item (e.g., a drug) has undergone secondary preparation within a pharmacy (e.g., mixing of a powder with a liquid or loading of a medication into a syringe), the method may allow a user to perform certain workflow functions that are only available on drugs with secondary preparation, or it may prevent the user from performing certain workflow functions that are available on drugs other than those with secondary preparation. In general, the term “secondary preparation” refers to any process that changes the state of an item as originally provided to a pharmacy into a different state to be used on a patient. In one example, suppose a drug and syringe have undergone secondary preparation by a process of loading the drug into the syringe. Under these circumstances, the method may inspect an item attribute (of the drug and/or the syringe) to determine that the drug is loaded into the syringe, and it may further inspect another item attribute to determine how long ago the drug was loaded into the syringe. If the syringe was prepared too long ago, the method may modify the workflow to prevent the syringe from being used on a patient. Still other examples of workflow variations corresponding to step S220 are described below with reference to FIGS. 3 through 9.

FIG. 3 is a workflow diagram illustrating a method 300 of blocking the distribution of a pharmacy kit containing unverified items, according to an embodiment of the inventive concept. In the method of FIG. 3, certain steps are performed through actions by a pharmacy technician and certain steps are performed through actions by a pharmacist. Alternatively, however, the steps could be performed by two different technicians, two different pharmacists, or two other types of pharmacy professionals, as provided by relevant laws, regulations, and/or institutional policies.

Referring to FIG. 3, the method begins with the pharmacy technician loading unverified pharmacy items (e.g., medications) into a kit and scanning the kit (S305). This loading step typically occurs within a hospital pharmacy as part of an inventory stocking process, and the scanning typically involves applying a bar code scanner or RFID scanner to packaging or labeling materials affixed to items in the kit. In general, the scanning process may capture information associated with individual pharmacy items, as well as the kit in general. It may also store the information in a database, e.g., within system 100. This information can include, e.g., descriptive attributes (e.g., National Drug Codes (NDCs) or Universal Product Codes (UPCs)) and contextual attributes, such as verification status, current or prior location, current or prior handler, lot number, and expiration date, and so on.

In the illustrated example, the scanning process is used to determine whether the items and/or kit have been verified by an authorized professional. This can be accomplished by comparing the captured information with previously stored information, such as one or more contextual attributes associated with the items and/or kit. Upon successful verification, the items and/or kit may be deployed to a location within a hospital or other facility. Upon failed verification, a warning is displayed on a screen to indicate the failure, and the user is advised that the distribution of the items cannot proceed (S310). In other words, the workflow is blocked.

Following the warning, a pharmacist may visually review unverified items and labels among those that were previously scanned (S315). This review process may be used to confirm that the items have the correct contents and labeling. The review process is deemed successful if the pharmacist is able to provide positive confirmation of the items as presented, or to fix any deficiencies that exist. In addition to inspecting the items themselves, the pharmacist may also compare the items with information stored in a computer system. For instance, the pharmacist may inspect a displayed list of recorded items and compare the list to the items actually presented.

Next, assuming a successful review process, the pharmacist provides formal verification that the items and/or kit have correct contents and labeling (S320). This formal verification may involve, for instance, entering data in a computer system or signing papers. Typically, such verification will indicate the pharmacist's identity, as well as the circumstances of the verification, such as time, date, location, methodology, etc.

Following verification, the technician may re-scan and deploy the kit for use in the hospital or other facility (S325). Because the kit has been verified at this point, the scanning does not produce an error or warning, and the workflow is not blocked. Finally, following use of the kit, it can be re-stocked by the technician (S330). The restocking generally introduces unverified items into the kit, so it may result in a return to step S305.

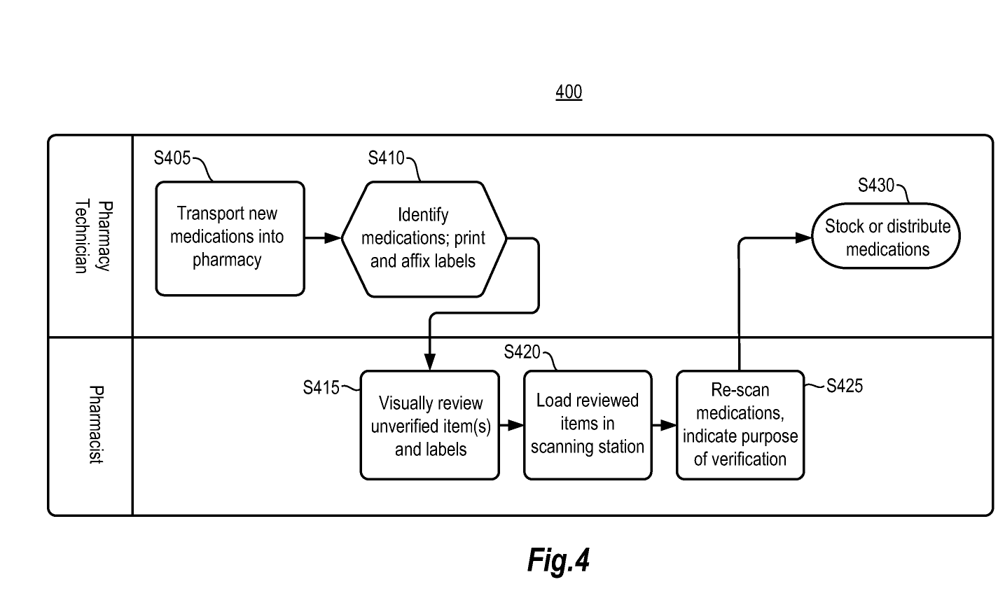

FIG. 4 is a workflow diagram illustrating a method of verifying multiple pharmacy items in batch mode, according to an embodiment of the inventive concept. This method is similar to that of FIG. 3 in that certain steps are performed by a technician while others are performed by a pharmacist. It differs from the method of FIG. 3 in that it involves the preparation and verification of individual items rather than kits.

Referring to FIG. 4, the technician transports or receives new items (e.g., medications) into a pharmacy (S405), and then identifies and labels the items (S410). The identification and labeling may involve automated processes, such as scanning existing barcode labels and/or printing and affixing RFID labels, although such processes are generally supervised by the technician to ensure accuracy. In this regard, step S410 can be considered a first step of a two-step verification process, i.e., a process involving two different professionals. Additionally, the scanning and labeling is typically accompanied by operations for storing relevant information in a computer system. For instance, when an item is identified by scanning, the scanned information may be added to a database of inventory. The database may also store known contextual attributes associated with the scanned item.

Next, the pharmacist visually reviews the items and corresponding labels to confirm their accuracy (S415). This step is similar to step S315 of FIG. 3, and it may likewise require the pharmacist to address any inaccuracies that are found in the course of review. Upon successful review, the items are loaded into a scanning station, e.g., an RFID scanning station (S420) and then re-scanned (S425). Collectively, steps S415 through S425 constitute a second step of the two-step verification process. Prior to this second step, the items are considered to be unverified because they have not completed the two-step verification process.

When performing the re-scanning in step S425, the pharmacist may provide input to a computer system to indicate that the re-scanned items have been formally verified for accuracy. Such input may be provided, for instance, through a user interface having a functional relationship with the scanning process. Additional inputs may be similarly provided to the computer system at various steps of the illustrated method to indicate other contextual attributes of the items being handled or otherwise processed. For instance, inputs may be provided to indicate who is handling the items, where the handling is being performed, and so on.

After the items have been verified by the two-step verification process, those items may be added to pharmacy stock or distributed within a hospital or other facility (S430).

FIG. 5 shows a display interface 500 confirming verification of a pharmacy item, according to an embodiment of the inventive concept. This interface provides examples of information that may be presented, captured, or otherwise used in the method of FIG. 3 or FIG. 4. For example, the display interface of FIG. 5 could be presented after re-scanning is performed in step S325 or S425 of FIG. 4 or 5, respectively.

Referring to FIG. 5, display interface 500 contains the following general information for a pharmacy item that has been verified: the item's name, NDC, and lot number. It also shows values for the following contextual attributes associated with the item: expiration date, verification status, verifying party (“Verified By”), verification method, and verification location (“Verified At”). As indicated by the “Verification Status” attribute, the item is “Verified”. Accordingly, the interface may be presented as part of process in which workflow is not blocked.

The attribute values shown in FIG. 5 are typically populated through manual data entry or by an automated process such as item scanning. These values can be entered, for instance, by a technician when preparing items in step S305 or S410, and they can be reviewed or updated, for instance, by a pharmacist when performing verification in step S315 or S415.

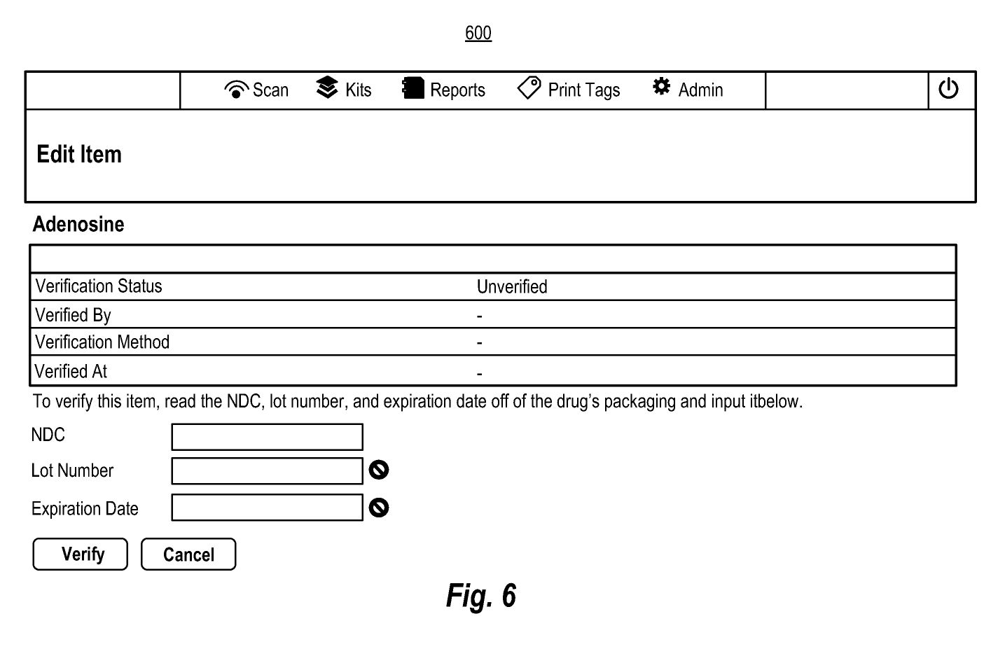

FIG. 6 shows a display interface 600 allowing input to verify a pharmacy item, according to an embodiment of the inventive concept. This interface can be presented to a user for manual and/or automated data entry, e.g., in step S305 or S410.

Referring to FIG. 6, display interface 600 contains the item name and shows contextual attributes, similar to display interface 500, but it is missing the item's NDC and values for the contextual attributes. As indicated above, the missing information can be entered into provided fields either by manual or automated data entry, e.g., in steps S305 or S410. Basic instructions for entering the missing information are shown in display interface 600. Once the missing information is entered into the fields, an authorized professional may verify the pharmacy item by pressing a button labeled “Verify”. This button may be pressed, for instance, by a pharmacist when performing verification in step S315 or S415. Doing so would change the value of the “Verification Status” attribute from “Unverified” to “Verified”.

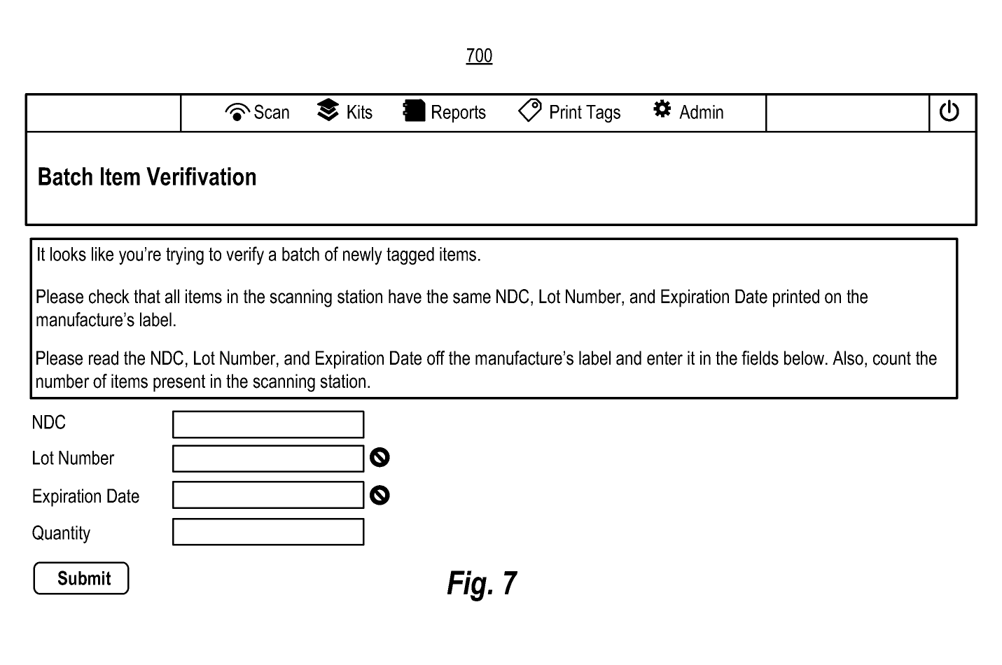

FIG. 7 shows a display interface 700 allowing input to verify multiple pharmacy items in batch mode, according to an embodiment of the inventive concept. Display interface 700 is similar to display interface 600, except that it contains instructions for multiple items rather than a single item, and it also allows a user to enter a quantity of an item.

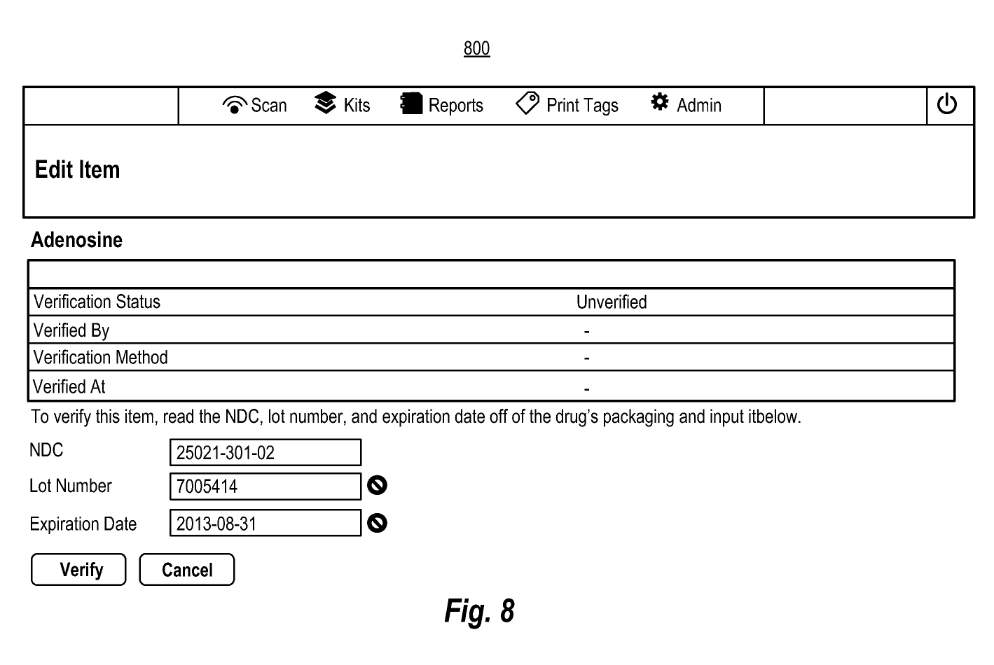

FIG. 8 shows a display interface 800 with information used to verify a pharmacy item, according to an embodiment of the inventive concept. Display interface 800 is the same as display interface 600, except that a user has entered an NDC, lot number, and expiration date for an item yet to be verified.

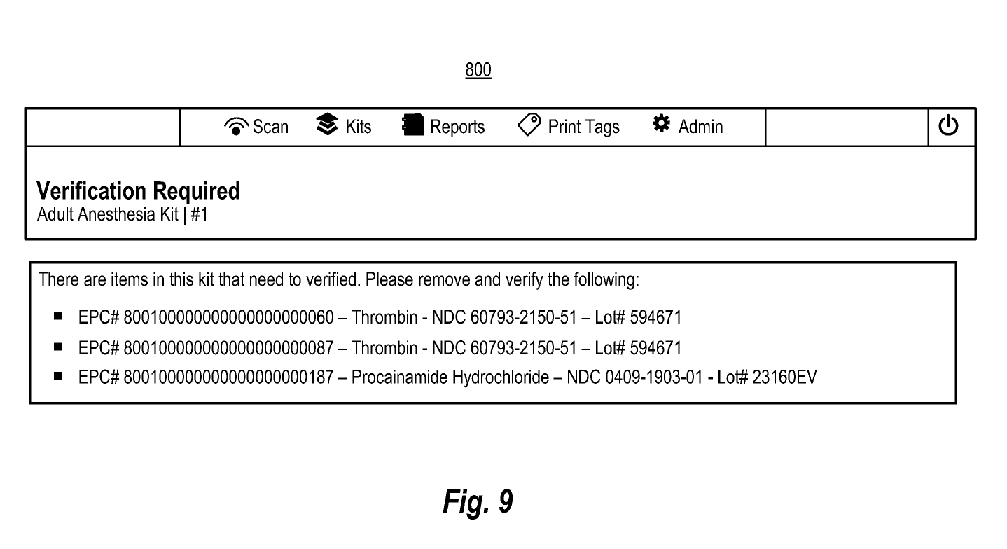

FIG. 9 shows a display interface 900 providing a warning that a scanned pharmacy kit contains unverified items, according to an embodiment of the inventive concept. This interface can be presented to a user when a workflow is blocked due to the presence of unverified items. For example, it could be presented to a user in step S310.

Referring to FIG. 9, display interface 900 includes a warning that a kit contains certain unverified items. It identifies those items by name as well as identifying numbers, and it provides instructions for verifying those items. Such verification could be performed through display interface 600, for example.

As indicated by the foregoing, pharmacy inventory management often requires multiple individuals reviewing and verifying an item is correctly tagged before it can be safely and accurately incorporated into the supply chain. The described embodiments provide systems and methods to guide users towards the appropriate tools to manage incompletely documented pharmaceutical products and to bar users from including medications that have not been verified as being accurately labeled from being deployed outside of the pharmacy where they could cause harm or waste. By using these and other embodiments, a hospital pharmacy may allow medications to be deployed with relative safety and accuracy throughout the hospital.

The foregoing is illustrative of various embodiments and is not to be construed as limiting the inventive concept. Although a few embodiments have been described, those skilled in the art will readily appreciate that many modifications are possible in the embodiments without materially departing from the novel teachings and advantages of the inventive concept. Accordingly, all such modifications are intended to be included within the scope of the inventive concept as defined in the claims.

What is claimed is:

1. A method of operating an electronic system, comprising:
receiving an input identifying one or more pharmacy items;
accessing stored information corresponding to the one or more pharmacy items based on the received input;
determining a value of at least one contextual attribute of the one or more pharmacy items based on the accessed information; and
selectively executing, blocking, or modifying a workflow to control distribution of the one or more pharmacy items according to the value of the at least one contextual attribute.

2. The method of claim 1, wherein the at least one contextual attribute indicates whether labeling and contents of the one or more pharmacy items have been verified by an authorized professional.

3. The method of claim 2, wherein the at least one contextual attribute indicates whether the labeling and contents of the one or more pharmacy items have been reviewed by at least two different pharmacy professionals.

4. The method of claim 3, wherein the at least two different pharmacy professionals comprise a first professional that prepared and labeled the or more pharmacy items and a second professional that authorizes distribution of the pharmacy items to an intended location.

5. The method of claim 3, wherein the at least two different pharmacy professionals comprise a first professional having a first level credential and a second professional having a second level credential higher than the first level credential.

6. The method of claim 2, wherein the at least one contextual attribute indicates whether the labeling and contents of the one or more pharmacy items have been reviewed by at least one pharmacy professional having a designated credential.

7. The method of claim 2, wherein the at least one contextual attribute indicates whether the labeling and contents of the one or more pharmacy items have been reviewed by at least two different pharmacy professionals including a last reviewer having a designated credential.

8. The method of claim 1, wherein the at least one contextual attribute indicates a current or prior location of the one or more pharmacy items.

9. The method of claim 1, wherein the at least one contextual attribute indicates a current or prior handler of the one or more pharmacy items.

10. The method of claim 1, wherein the one or more pharmacy items constitute a pharmacy kit and the at least one contextual attribute corresponds to the pharmacy kit.

11. The method of claim 1, further comprising receiving an additional input to modify at least one contextual attribute of the one or more pharmacy items.

12. The method of claim 11, wherein the additional input modifies the at least one contextual attribute to indicate that the one or more pharmacy items have been verified.

13. The method of claim 1, further comprising modifying a display to indicate the selective executing, blocking, or modifying the workflow.

14. The method of claim 13, wherein selectively blocking the workflow comprises the display identifying unverified pharmacy items.

15. The method of claim 13, wherein selectively blocking the workflow comprises the display prompting a user to verify unverified items.

16. The method of claim 1, wherein receiving the input comprises scanning one or more radio-frequency identification (RFID) tags associated with the one or more items.

17. The method of claim 1, wherein executing the workflow comprises printing a list of drugs to be sent out with a kit, where the contextual attribute indicates that the one or more pharmacy items are verified, and blocking the workflow comprises preventing the list from being printed where the contextual attribute indicates that the one or more pharmacy items are unverified.

18. The method of claim 1, wherein selectively executing, blocking, or modifying the workflow comprises printing a list of drugs to be sent out with a kit where the one or more pharmacy items are verified and blocking printing of the list where the one or more pharmacy items are unverified.

19. The method of claim 1, wherein selectively executing, blocking, or modifying the workflow comprises selectively displaying one of multiple distinct screens to a user according to according to the value of the at least one contextual attribute.

20. The method of claim 1, wherein selectively executing, blocking, or modifying the workflow comprises blocking a user from performing a next step of the workflow and/or displaying a warning where the value of the at least one contextual attribute indicates that a drug is expired or recalled.

21. The method of claim 1, wherein selectively executing, blocking, or modifying the workflow comprises displaying information indicating the presence of a new patient and/or preventing a user from administering a drug to the new patient where the value of the at least one contextual attribute indicates that the drug has already been used on another patient.

22. The method of claim 1, wherein selectively executing, blocking, or modifying the workflow comprises allowing or disallowing a workflow function contingent upon whether the one or more pharmacy items have undergone secondary preparation within the pharmacy.

23. The method of claim 1, wherein selectively executing, blocking, or modifying the workflow comprises displaying a warning where the at least one contextual attribute indicates that a drug is considered to be a substitute for another drug or has abnormal strength.

24. A system, comprising:
an input capture unit configured to receive an input identifying one or more pharmacy items;
a contextual attribute identification unit configured to access stored information corresponding to the one or more pharmacy items based on the received input, and further configured to determine a value of at least one contextual attribute of the one or more pharmacy items based on the accessed information; and
a workflow control component configured to selectively execute, block, or modify a workflow to control distribution of the one or more pharmacy items according to the value of the at least one contextual attribute.

25. The system of claim 24, further comprising a display component configured to selectively display one of multiple distinct screens to a user according to according to the value of the at least one contextual attribute.

26. The system of claim 24, wherein the at least one contextual attribute indicates whether labeling and contents of the one or more pharmacy items have been verified by an authorized professional.

27. The system of claim 26, wherein the workflow control component blocks the workflow where the at least one contextual attribute indicates that labeling and contents of the one or more pharmacy items have not been verified by an authorized professional.
